(19) United States
(12) Patent Application Publication
(10) Pub. No.: US 2025/0276158 A1
(43) Pub. Date: Sep. 4, 2025

(54) BALLOON-BASED FLOW RESTRICTORS FOR ENDOVASCULAR USE AND RELATED METHODS

(71) Applicant: Bard Peripheral Vascular, Inc., Franklin Lakes, NJ (US)

(72) Inventors: Andrzej J. Chanduszko, Chandler, AZ (US); Matt R. Casiraro, Tempe, AZ (US); Alexander W. Tessmer, Phoenix, AZ (US); Eric A. Fallows, Apex, NC (US)

(21) Appl. No.: 18/858,021

(22) PCT Filed: Apr. 18, 2022
(86) PCT No.: PCT/US2022/025183

§ 371 Date: Oct. 26, 2023

(51) Int. Cl. A61M 25/10

(52) U.S. Cl. CPC A61M 25/1011; A61M 25/1002; A61M 2025/1052; A61M 2025/1059; A61M 2025/1061; A61M 2025/1081; A61M 2210/12

(57) ABSTRACT

An apparatus for temporarily limiting blood flow to heart uses balloon-based technologies to vary a diameter of the outflow aperture of an associated vessel. Related methods are also disclosed.

### BACKGROUND OF THE INVENTION

Congestive heart failure is a condition in which the heart cannot pump enough blood to meet the body's needs. When the heart's blood pumping ability decreases, the kidneys cannot work as they should, which leads to an excess of fluid in the body, termed “hypervolemia.” Symptoms may vary depending on where the fluid is collecting and what other health problems are present, but commonly include unexplained and rapid weight gain, swelling in the arms and legs, abdominal swelling (which is common with liver problems), and shortness of breath caused by fluid in the lungs.

Currently, there are several approaches to treatment for hyper-volemia. One of the most common treatments involves the use of diuretics, which are drugs that increase the amount of urine the body produces, which in turn reduces the fluid overload. While sometimes helpful, some patients do not respond adequately to diuretic therapy. By temporarily reducing blood flow to heart the symptoms of fluid overload can be improved, leading to increased diuretic efficiency and further improvement to patient's condition. This may be achieved by deploying a balloon into the Superior Vena Cava (SVC). However, because this creates a full occlusion, the balloon needs to be deflated repeatedly during treatment to prevent excessive blood pressure in the brain, which adds to the complexity of the procedure and does not afford a long-term solution.

Accordingly, a need is identified for an apparatus for temporarily limiting blood flow to heart, such as by using balloon-based technologies that vary the diameter of the outflow aperture of an associated vessel. The advantage of the proposed concept is that the treatments can control the degree of occlusion due to design features of the balloons, and thus avoid the problems with full occlusion noted above, and perhaps resolve other issues that remain to be discovered.

### SUMMARY OF THE INVENTION

An object of the disclosure is to provide an apparatus for endovascular use that may temporarily limit blood flow to heart using balloon-based technologies to vary the diameter of the outflow aperture of an associated vessel.

According to a first aspect of the disclosure, an apparatus for selectively occluding a vessel comprises a catheter shaft including a first inflation lumen and a second inflation lumen. The apparatus further includes a first group of inflatable balloons in communication with the first inflation lumen and a second group of inflatable balloons in communication with the second inflation lumen. By selectively or differentially inflating one group of the balloons as compared to other of the second group of balloons, the apparatus may vary the diameter of the outflow aperture of an associated vessel and temporarily limit fluid flow, such as from the associated vessel to the heart.

In one embodiment, wherein the first and second groups of inflatable balloons comprise peripheral balloons surrounding a central balloon. The catheter shaft may further include a third inflation lumen in communication with the central balloon. Alternatively, the central balloon may be in communication with the first inflation lumen.

A further aspect of the disclosure pertains to an apparatus for selectively occluding a vessel. The apparatus comprises a catheter shaft supporting a plurality of balloons in a single cross-section of the catheter, at least one of the plurality of balloons having a generally spherical shape. The balloons may comprise one or more compliant balloons. The balloons may comprise a central balloon surrounded by one or more peripheral balloons.

In one embodiment, the catheter shaft includes a first inflation lumen and a second inflation lumen. At least a first one of the balloons may be in communication with the first inflation lumen. At least a second one of the balloons may be in communication with the second inflation lumen.

Yet another aspect of the disclosure relates to an apparatus for forming a catheter for selectively occluding a vessel. The apparatus comprises a catheter shaft supporting a plurality of balloons in a single cross-section. At least one of the balloons comprises a tapered balloon having an apical region adapted to create a minimal point of contact with an inner wall of the vessel.

The at least one balloon may taper continuously from a proximal end portion forming the apex to a distal end portion. The catheter shaft may include a first inflation lumen and a second inflation lumen. At least a first one of the plurality of balloons may be in communication with the first inflation lumen and at least a second one of the plurality of balloons may be in communication with the second inflation lumen.

Still a further aspect of the disclosure relates to an apparatus for selectively occluding a vessel. The apparatus comprises a catheter shaft supporting an inflatable perfusion balloon. A flow restrictor is provided external to the inflatable perfusion balloon for selectively moving in a longitudinal direction relative to the inflatable perfusion balloon for restricting flow therethrough when the inflatable perfusion balloon is inflated.

In one example, the flow restrictor comprises a cone for at least partially covering and obstructing a proximal end of the perfusion balloon.

The perfusion balloon may comprise a plurality of balloons in a single cross-section. The flow restrictor may be supported by a sheath overlying the catheter shaft.

Yet another aspect of the disclosure pertains to an apparatus for forming a catheter for selectively occluding a vessel. The apparatus comprises a catheter shaft supporting a plurality of inflatable balloons in tandem. Each of the plurality of balloons has a different nominal diameter when inflated.

In one example, the plurality of balloons comprise a first proximal balloon having a first diameter and a second distal balloon having a second nominal diameter smaller than the first nominal diameter. The catheter shaft includes a first inflation lumen and a second inflation lumen. At least a first one of the plurality of balloons may be in communication with the first inflation lumen and at least a second one of the plurality of balloons may be in communication with the second inflation lumen.

In this or a different example, a third balloon is intermediate of the first and second balloons. The third balloon includes a third nominal diameter intermediate of the first and second diameters. The catheter may further include a first inflation lumen, a second inflation lumen, and a third inflation lumen. At least a first one of the plurality of balloons may be in communication with the first inflation lumen, at least a second one of the plurality of balloons may be in communication with the second inflation lumen, and at least a third one of the plurality of inflatable balloons may be in communication with the third inflation lumen.

This disclosure also pertains to an apparatus for selectively occluding first and second branched vessels. The apparatus comprises a main catheter shaft comprising first and second inflatable balloons for independently occluding the first and second branched vessels. In one example, a first auxiliary shaft at least partially within the main catheter shaft supports the first inflatable balloon and a second auxiliary shaft at least partially within the main catheter shaft supports the second inflatable balloon. The first and second balloons may be serially arranged or arranged in parallel.

Another aspect of the disclosure relates to an apparatus for selectively occluding a vessel. The apparatus comprises a catheter shaft including an inflatable balloon having an annular shape. A flexible cone interconnects a portion of the catheter shaft and an end portion of the at least one inflatable balloon.

In one example, the flexible cone comprises a proximal cone connecting a proximal portion of the shaft with a proximal end portion of the annular inflatable balloon. A distal cone may be connected between a distal portion of the shaft and a distal end of the annular inflatable balloon. The catheter shaft may pass through a central opening of the annular inflatable balloon.

In any disclosed embodiment, the apparatus may include a controller for controlling an inflation status of the one or more balloons. A sensor for sensing a parameter of the fluid flow in the vessel may be located adjacent a distal end of the catheter shaft, such as distal of the one or more balloons, or proximal to the one or more balloons. The sensor may comprise a first proximal sensor and a second distal sensor.

A further aspect of the disclosure pertains to a method for selectively occluding a vessel. The method includes selectively inflating a first group inflatable balloons in the vessel independently of a second group of inflatable balloons. The inflating step may comprise inflating one or more peripheral balloons as the first group, which surround a central balloon. The method may further comprise inflating the central balloon, or selectively moving a flow restrictor toward or away from a proximal end of the inflatable balloons. The second group may also surround or include the central balloon.

Yet another disclosed aspect pertains to a method for selectively occluding a vessel. The method includes moving a flow restrictor external to a perfusion balloon in a longitudinal direction relative to the inflatable perfusion balloon in order to restrict flow therethrough when the inflatable perfusion balloon is inflated.

Still another aspect of this disclosure pertains to a method for selectively occluding a vessel. The method includes selectively inflating a plurality of inflatable balloons connected to a catheter shaft in tandem, each of the plurality of balloons having a different diameter when inflated.

A further disclosed aspect is a method for selectively occluding first and second branched vessels. The method includes positioning a main catheter shaft comprising first and second inflatable balloons for independently partially occluding each of the first and second branched vessels. The method may further include positioning a first auxiliary shaft at least partially within the main catheter shaft supporting the first inflatable balloon in the first branch vessel and positioning a second auxiliary shaft at least partially within the main catheter supporting the second inflatable balloon in the second branch vessel.

Yet another disclosed aspect pertains to a method for selectively occluding a vessel. The method comprises expanding a flexible cone inter-connecting an end portion of a catheter shaft and an end portion of at least one inflatable balloon having an annular shape. In one example, the flexible cone is a proximal cone, and the method further includes expanding a distal cone connected between a distal portion of the shaft and a distal end of the at least one inflatable balloon.

Any of the foregoing methods may comprise the step of controlling an inflation status of the apparatus based on a sensed parameter of fluid flow within a vessel.

### BRIEF DESCRIPTION OF THE DRAWINGS

The above and further advantages of the present disclosure may be better understood by referring to the following description in conjunction with the accompanying drawings in which:

FIG. 1 is a side view of a balloon-based flow restrictor located in a vessel.

FIG. 1A is a cross-sectional view along line 1A-1A of FIG. 1.

FIG. 1B is a schematic view of a cross-section taken along line 1B-1B of FIG. 1.

FIG. 1C is an enlarged cross-sectional view along line 1C-1C of FIG. 1.

FIG. 2 is a side view of an alternative embodiment of balloon-based flow restrictor.

FIG. 3 is yet another embodiment of a balloon-based flow restrictor.

FIGS. 4 and 5 illustrate a further embodiment of a balloon-based flow restrictor in alternative positions.

FIG. 6 is yet another embodiment of a balloon-based flow restrictor.

FIGS. 7 and 8 illustrate further embodiments of balloon-based flow restrictors.

FIGS. 9 and 10 illustrate alternative positions of further embodiments of balloon-based flow restrictors.

FIGS. 9A and 10A are cross-sectional views taken along lines 9A-9A of FIGS. 9 and 10A-10A of FIG. 10, respectively.

FIGS. 11 and 12 illustrate still a further embodiment of balloon-based flow restrictors.

The dimensions of some of the elements may be exaggerated relative to other elements for clarity or several physical components may be included in one functional block or element. Further, sometimes reference numerals may be repeated among the drawings to indicate corresponding or analogous elements. Moreover, some of the parts depicted in the drawings may be combined into a single function.

### DETAILED DESCRIPTION

In the following detailed description, numerous specific details are set forth to provide a thorough understanding of the presently disclosed invention(s). The disclosed embodiments may be practiced without these specific details. In other instances, well-known methods, procedures, components, or structures may not have been described in detail so as not to obscure the disclosed inventive concepts.

The invention of this disclosure is not limited in its application to the details of construction and the arrangement of the components set forth in the following description or illustrated in the drawings. The inventive concepts disclosed are capable of other embodiments or of being practiced or carried out in various ways. It is also to be understood that the phraseology and terminology employed herein are for the purpose of description and should not be regarded as limiting.

Certain features of the disclosed embodiments that are, for clarity, described in the context of separate embodiments, may also be provided in combination in a single embodiment. Conversely, various features of the invention that are, for brevity, described in the context of a single embodiment, may also be provided separately or in any suitable sub-combination.

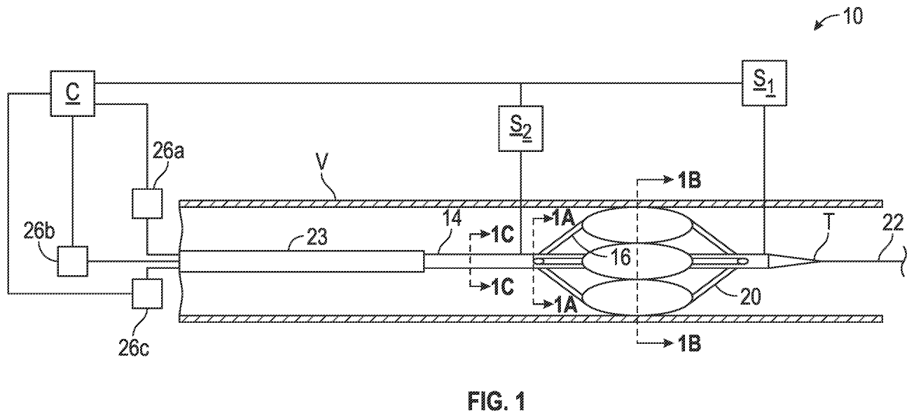

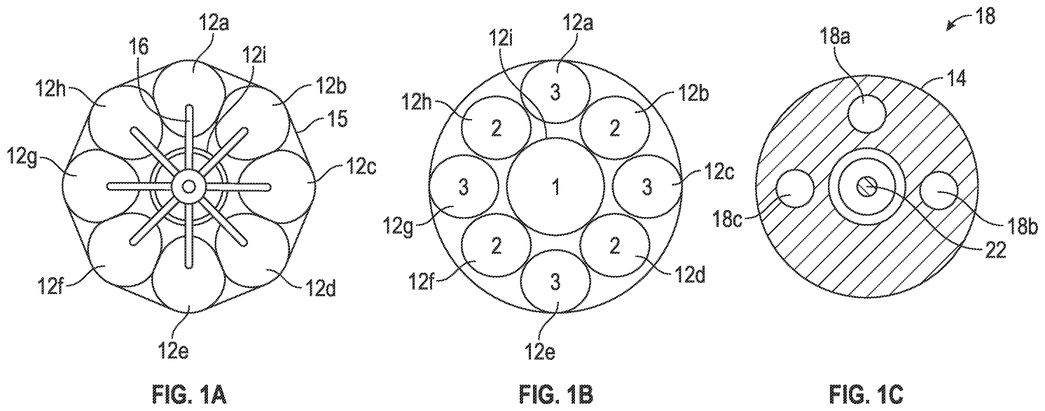

With reference to FIG. 1, provided is an apparatus in the form of a catheter 10 having a distal end portion including an expandable or inflatable perfusion balloon 12 mounted on a catheter shaft 14. The balloon 12 in the illustrated embodiment comprises a plurality of independently inflatable balloons (or cells) 12a-12i arranged in a single cross-section, as can be understood from the cross-sectional views of FIGS. 1A and 1B. The individual balloons 12a-12i may be generally oblong in shape with a generally cylindrical central portion intermediate tapered or conical ends. The balloons 12a-12i may directly connect to each other at points of external contact, such as by adhesive or the like. Additionally or alternatively, an external shell 15 (shown in FIG. 1A only) may surround and hold the balloons 12a-12i together as a single unit for movement together with the associated shaft 14.

As can be understood, the perfusion balloon 12 may comprise outer balloons 12a-12h peripherally arranged and partially or fully surrounding at least one inner or central balloon 12i. While nine total balloons are shown in this example, any number greater than four may be used (e.g, two groups of two balloons), such as 5, 6, 7, 8, 10, or up to 20 balloons. The central or inner balloon 12i may even be omitted in some circumstances while achieving the desired selective occlusion, as outlined further in the following description. Moreover, it is also possible to provide more than one inner or central balloon 12i, or to omit any one or more of the outer balloons 12a-12h.

Some or all of the balloons 12a-12i may be non-compliant, meaning the continued application of fluid pressure to the interior thereof does not meaningfully change the outer dimension or diameter thereof (which is typically achieved by using a layer of inelastic fibers applied to a blow-molded base balloon layer, possibly in combination with adhesives and additional layers). Alternatively, some or all of the balloons 12a-12i may be compliant or semi-compliant, meaning added fluid pressure to the interior will result in a further increase in diameter (more increase for compliant than semi-compliant). A combination of different types of compliance may also be provided by forming the balloons 12a-12i of different materials. In one particular example, the inner balloon 12i is compliant (e.g., comprised of an elastic material), while the remainder of the balloons 12a-12h are non-compliant (e.g., comprised of an inelastic material (e.g., fibers)). The outer shell 15 may also be made non-compliant to constrain the maximum diameter of balloon 12, but could be compliant or semi-compliant as well.

The outer balloons 12a-12h may each be individually inflated by a proximal neck 16 associated therewith. The proximal neck 16 of each outer balloons 12a-12h is tubular and may communicate with one or more inflation lumens 18 to supply a fluid, such as saline, to an interior compartment of the balloon to achieve inflation. As will be outlined in further detail below, the inflation lumens 18 may extend within the shaft 14 to communicate with a corresponding number of the proximal necks 16 to achieve selective inflation of the balloons 12a-12h.

The outer balloons 12a-12h may also include distal necks 20 for connecting to the shaft 14 in a sealed condition proximal of a distal tip T. This tip T may include a distal opening through which a guidewire 22 passes from a guidewire lumen 24 within the shaft 14 commencing at the proximal end thereof. Markers visible from outside the body (e.g., radiopaque markers visible using fluoroscopy) may be provided as desired or necessary to locate the catheter 10 (such as on the shaft 14, the perfusion balloon(s) 12, or both).

The inner or central balloon(s) 12i may surround and be directly supported by the shaft 14. These inner balloon(s) 12i may be inflated by a separate inflation lumen 18a provided in the shaft 14, or by either of the inflation lumens 18b, 18c. In any case, the selected inflation lumen 18a, 18b, 18c may communicate via an opening (not shown) into the interior compartment of the balloon 12i.

As perhaps best understood in FIG. 1B, the distinct groups of the outer balloons 12a-12h may communicate with different inflation lumens 18b, 18c in the shaft 14. For example, balloons 12b, 12d, 12f, 12h may communicate with inflation lumen 18b (labeled Group “2”), and balloons 12a, 12c, 12e, and 12g may communicate with inflation lumen 18c (labeled Group “3”). The groups may be alternating, as shown, to provide a known degree of flow control based on the inflation of each respective group, but could also be grouped differently (e.g., two adjacent balloons in one group, and a third balloon in another), or randomly grouped (e.g., balloons 12b, 12e could form one group, balloons 12a and 12h could form another group, and the other remaining balloons 12c, 12d, 12g, 12h could form one or more groups). Likewise, as noted, the inner or central balloon 12i may be independently inflated or deflated by a separate and distinct inflation lumen 18a.

The inflation fluid may be supplied by distinct indeflators 26a, 26b, 26c as shown in FIG. 1. One indeflator 26a, 26b, 26c may be connected to each lumen 18a, 18c, 18c by a port in a corresponding hub or other connector (not shown). Instead of multiple lumens 18a, 18b, 18c within a single shaft 14, it is also possible to use independent shafts with one or more inflation lumens (not shown), including possibly one shaft for supplying fluid to a selected number of the balloons (and possibly only one balloon in the case of inner balloon 12i).

Thus, as can be appreciated, the balloon 12 may be located in a vessel V, such as the Superior Vena Cava (SVC), with the guidance of a guide-wire 22 and a suitable sheath 23. By then controlling the inflation of different groups of the balloons 12a-12i, the occlusive or perfusive character of the balloon 12 may be selectively controlled in a precise and reliable matter, and without the need for total deflation to avoid full occlusion and re-inflation to achieve the desire flow restriction. For example, the balloons 12b, 12d, 12f, 12h of Group 1 may remain deflated while the balloons 12a, 12c, 12e, 12g of Group 2 are inflated (partially or fully), or vice-versa. The central or inner balloon 12i may be inflated (partially or fully) or deflated independently of or together with these groups, and when all balloons 12a-12i are inflated, there remain interstitial gaps or spaces that allow for continued flow. This provides the clinician with a great deal of flexibility in controlling the amount of fluid flow in vessel V by reducing the size of the outflow aperture in a selective manner.

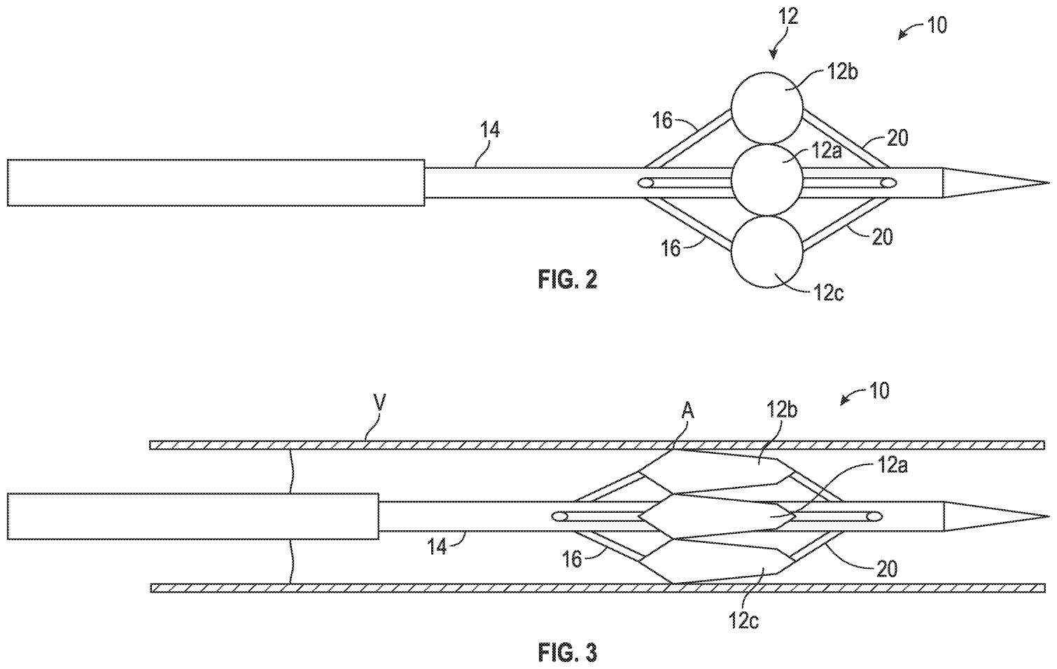

Further embodiments of a balloon-based endovascular flow restrictor are shown in FIGS. 2 and 3. In the FIG. 2 arrangement, catheter 10 may be substantially similar to the FIG. 1 version, except that at least one of the individual balloons or inflatable balloons 12 is generally spherical. The arrangement may comprise a central balloon 12a, which may or may not be generally spherical, surrounded by peripheral spherical balloons 12b, 12c (which may include necks 16, 20 forming a connection and in fluid communication with one or more inflation lumens 18a-18c of shaft 14, as shown in FIG. 1C). The spherical balloons generally create a smaller point of contact with the vessel wall, and also enhance smooth fluid flow. The generally spherical balloon(s) 12 may be compliant in nature. As with the previous embodiment, the balloons 12a, 12b, 12c (and others present) may be selectively inflated (fully or partially) or deflated, including in one or more groups, to achieve a desired degree of flow restriction.

Other shapes of balloons may also be used. For example, as shown in FIG. 3, one or more of the inflatable balloons may comprise a tapered balloon 12. The tapered balloon 12 includes an apical region or apex A extending in a radial direction and circumferentially around a portion thereof, the apex A forming the point of contact between the vessel and the tapered balloon 12 when inflated. This tapered balloon 12 may taper continuously from a proximal end portion forming the apex A to a distal end portion, and may be connected to shaft 14 by necks 16, 20, as per the above embodiments.

Likewise, a plurality of balloons 12 may be provided, and each may be similarly tapered. As shown in FIG. 1C, the associated catheter shaft 14 may include at least a first inflation lumen 18b and a second inflation lumen 18c, and at least a first one of the plurality of balloons 12, such as balloon 12b, is in communication with the first inflation lumen and at least a second one of the plurality of balloons, such as balloon 12c, is in communication with the second inflation lumen. A central or inner balloon 12a may be in communication with a further inflation lumen 18a in the shaft 14. Thus, as with the previous embodiments, the balloons 12a, 12b, 12c (and others present) may be selectively inflated (fully or partially) or deflated, including in one or more groups, to achieve a desired degree of flow restriction.

When the balloon 12 is thus inflated, this apical region or apex A creates a minimal point of contact between the balloon 12 or balloons 12a, 12b, 12c and an adjacent inner wall of the vessel V. The balloon(s) 12; 12a, 12b, 12c thus provide an occlusive effect, but may still allow perfusion through one or more central passages between the balloons 12a, 12c, 12c. The tapering of the outer or peripheral balloons 12b, 12c also helps to create interstitial spaces that allow for flow to expand once the passing through the portion of the balloons 12a, 12b, 12c including the apical region or apex A. This may serve to further minimize the occlusive effect while still providing a desired restriction depending on the size and number of the balloons used.

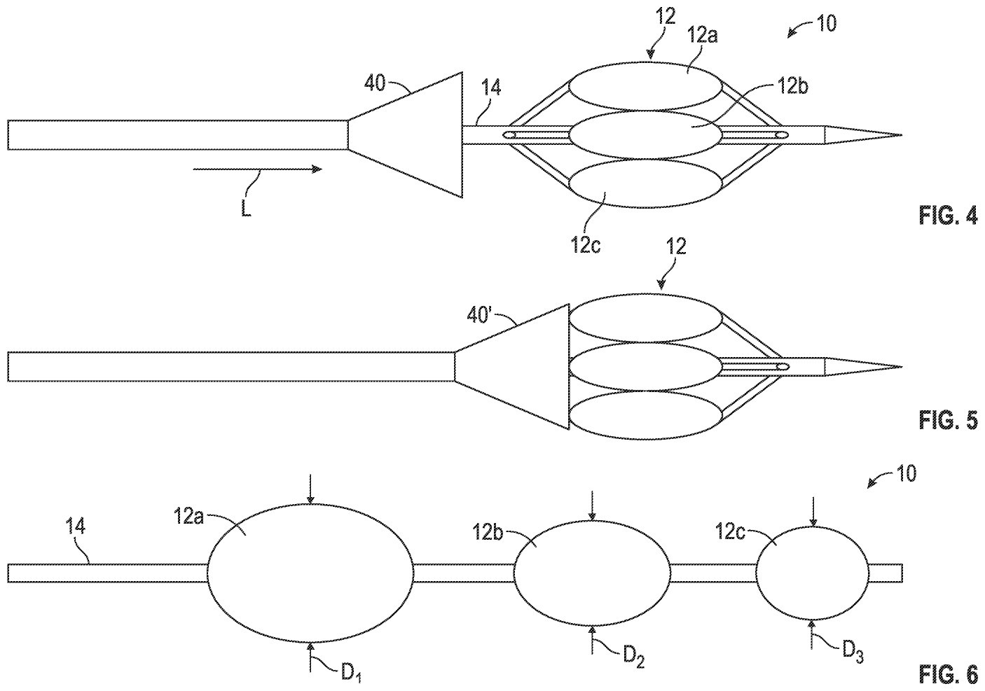

Another version of a balloon-based flow restrictor in the form of a catheter 10 for endovascular use is provided with reference to FIGS. 4 and 5. As with the above embodiments, a catheter shaft 14 supports an inflatable perfusion balloon 12, which may comprise a plurality of balloons 12a, 12b, 12c in a single-cross-section (e.g., vertical-see FIG. 1B). A flow restrictor 40 is provided external to the perfusion balloon 12 for selectively moving in a longitudinal direction L relative to the inflatable perfusion balloon for restricting flow therethrough when the inflatable perfusion balloon 12 is inflated (compare withdrawn position of restrictor 40 with deployed position 40′ in FIG. 5). By adjusting the position of the restrictor 40, which may be connected to a sheath 42 for receiving the catheter 10, the amount of occlusion may be controlled when the balloon 12 is inflated, as shown.

In the illustrated example, the flow restrictor 40 comprises a cone for covering and thus obstructing fluid flow entering a proximal end of the perfusion balloon 12. However, the restrictor 40 may take other forms or shapes as well, such as for example a hemispherical cup (not shown). The restrictor 40 may also be sized to have an outer diameter at a distal open end that is less than or greater than the diameter of the balloon 12 depending on the desired level of flow restriction to be achieved (the greater diameter serving to potentially occlude more flow than a lesser one).

Turning to FIG. 6, a further version of a catheter 10 for selectively occluding a vessel is provided. In this example, a catheter shaft 14 supports a plurality of inflatable balloons 12 in tandem. Each of the plurality of balloons 12 is formed having a different nominal diameter when inflated (with the “nominal” diameter referring to a given diameter of balloon expansion under a particular internal pressure, which diameter is usually used to rate balloons by size for use in various locations of the vasculature, depending on the size and nature of the procedure).

For example, the plurality of balloons may comprise a first proximal balloon 12a having a first nominal diameter D1 and a second distal balloon 12b having a second nominal diameter D2 smaller than the first diameter D1. Independent inflation may be achieved using suitable inflation lumens 18a, 18b in the shaft (see FIG. 1C). Optionally, a third balloon 12c may be provided having a third nominal diameter D3 smaller than the second diameter D2, and in communication with a third inflation lumen 18c (see FIG. 1C).

The nominal diameters of the balloons may be selected to provide a different amount of obstruction to the vessel. For example, the nominal diameter of the first balloon 12a may provide a 1-75% flow restriction, the intermediate balloon 12b a 1-50% restriction, and the third balloon a 1-25% restriction. These percentages are provided for exemplary purposes only, and may be varied to achieve a desired flow restriction (e.g., anywhere from 1-99% flow restriction per balloon). Additional balloons of similar or varying nominal diameters may also be provided.

As shown in FIG. 1C, the catheter shaft 14 may include a first inflation lumen 18a and a second inflation lumen 18b. A first one of the balloons 12a may be in communication with the first inflation lumen 18a and a second balloon 12b may be in communication with the second inflation lumen 18. Likewise, a third balloon 12c if present may communicate with a third inflation lumen 18c of the shaft 14, which may be the same construction as shown in FIG. 1C or as otherwise described.

Thus, as can be appreciated, the balloon 12 may be located in a vessel, such as the Superior Vena Cava (SVC), with the guidance of a guide-wire (not shown). By then controlling the inflation of different balloons 12a, 12b, 12c sequentially (e.g., smallest diameter to largest diameter), the occlusive or perfusive character of the balloon 12 may be selectively controlled in a precise and reliable matter, and without the need for total deflation to avoid full occlusion and re-inflation to achieve the desired flow restriction. This provides the clinician with a great deal of flexibility in controlling the amount of fluid flow in the vessel.

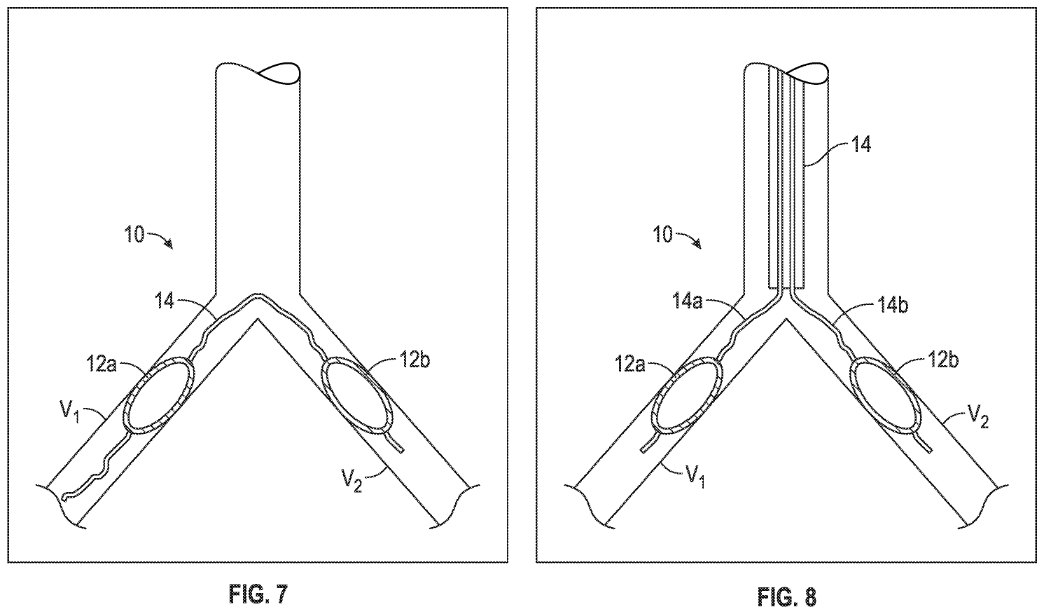

With further reference to FIGS. 7 and 8, an example of a catheter 10 for selectively occluding first and second branched vessels V1, V2, such as in the iliofemoral venous anatomy. In this example, a main catheter shaft 14 include first and second inflatable balloons 12a, 12b for indpendently occluding the first and second branched vessels V1, V2 when inflated. As can be appreciated, the balloons 12a, 12b are arranged serially on the shaft 14, and may be inflated independently by separate inflation lumens therein (not shown, but see FIG. 1C). Access may be achieved by way of femoral or popliteal vein, such as by using a guidewire (not shown).

In the FIG. 8 example, a first auxiliary catheter shaft 14a at least partially within the main catheter shaft 14 supports the first inflatable balloon 12a, and is independently maneuverable relative to the main catheter shaft. A second auxiliary catheter shaft 14b is at least partially within the main catheter shaft 14, and likewise independently maneuverable, supports the second inflatable balloon 12b. As can be appreciated, the balloons 12a, 12b are thus arranged in parallel for occluding the first and second branched vessels V1, V2 when inflated, which may be achieved independently by separate inflation lumens in the associated auxiliary shafts 14a, 14b. Access in this example may be achieved from the jugular or above the Inferior Vena Cava, again using suitable guide-wires (e.g., one for each auxiliary shaft 14a, 14b).

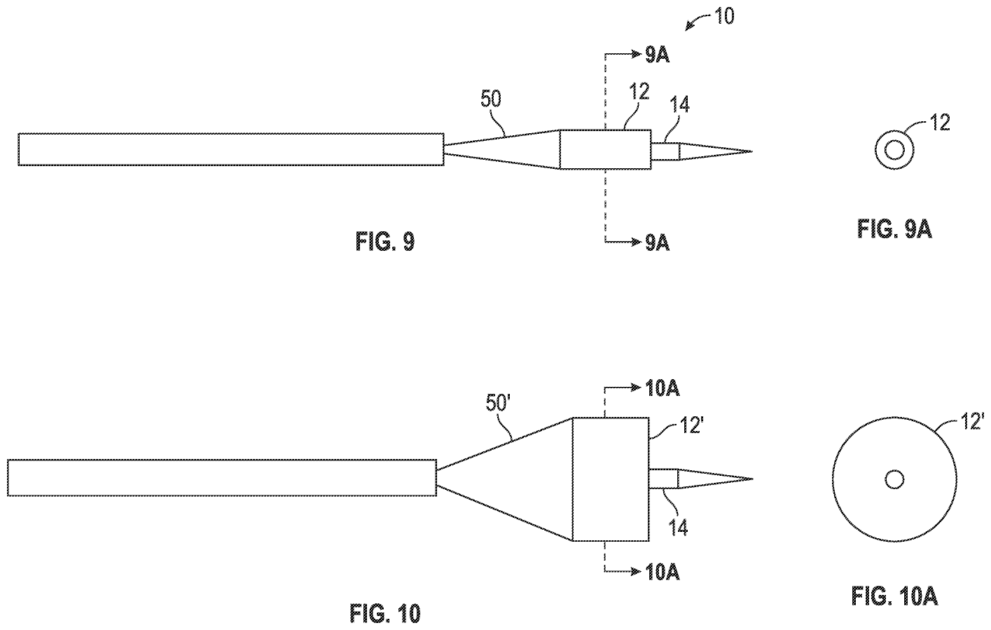

An example similar to the one shown in FIGS. 4 and 5 is provided in FIGS. 9, 10, 11, and 12. In the FIG. 9 example, a catheter 10 for selectively occluding a vessel includes a catheter shaft 14 with at least one inflatable balloon 12. This balloon 12 may be annular in shape, as shown in FIG. 9A (deflated), and in communication with an inflation lumen of the shaft 14. The shaft 14 thus extends through a central opening or passage of the annular balloon 12.

At a proximal end of the balloon 12, a flexible cone 50 interconnects a proximal end portion the shaft 14 and a proximal end portion of the at least one inflatable balloon 12. Thus, when the balloon 12 is expanded (note balloon 12′ in FIGS. 10, 10A), the flexible cone 50 expands (note cone 50′ in FIG. 10) and thus provides for the desired selective occlusion in a gradual, consistent manner while inflation progresses. The presence of the cone 50 at the proximal end also aids in preventing turbulent flow.

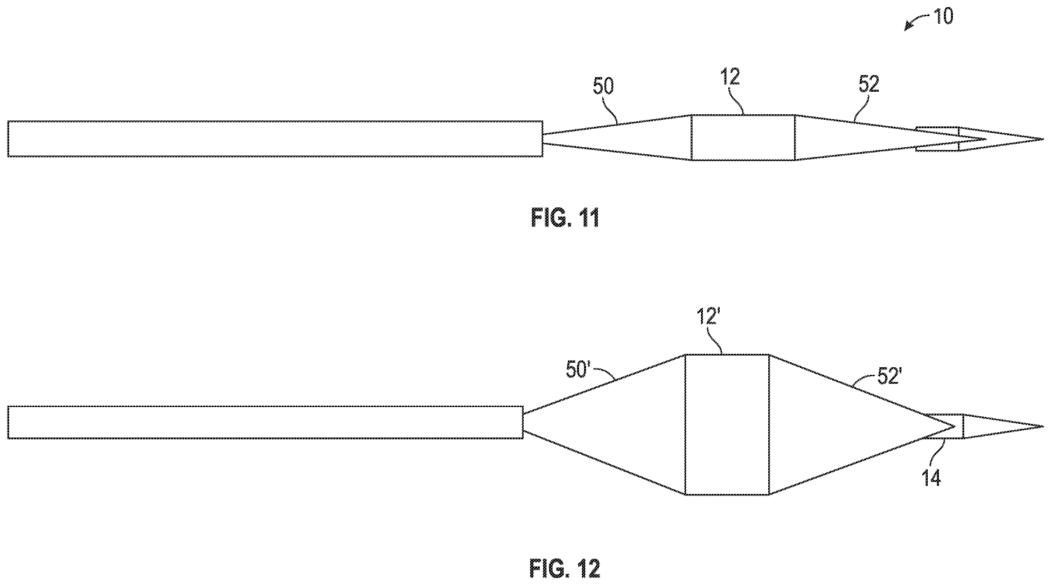

An alternative is shown in FIG. 11, which further includes a distal flexible cone 52 connecting a distal end of the balloon 12 with the shaft 14. The presence of both cones 50, 52 when expanded with balloon 12 (note 12′, 50′, 52′ in FIG. 12) further aids in achieving smooth, non-turbulent flow.

In any of the foregoing embodiments, the catheter 10 may further comprise a controller C for controlling an inflation status of the associated balloon(s) 12. The controller C may receive signals from a sensor S1 located adjacent a distal end of the catheter 10, such as adjacent to the catheter tip T in FIG. 1 (or at least distal of the balloon 12) for sensing a parameter of the fluid flow in the vessel, such as for example pressure. The sensor S1 may be used to provide a signal to the controller C, which may then control the inflation status of one or more of the balloons 12 (such as by regulating the associated indeflator 26a-26c in the example of FIG. 1).

Additionally or alternatively, a sensor S2 may also be optionally provided proximal to the balloon 12 for sensing a parameter of the fluid flow in vessel, such as pressure. By comparing the outputs of sensors S1, S2 measuring flow characteristics both upstream and downstream of the catheter 10 (which outputs may reflect common parameters, such as pressure), the expansion of one or more of the balloon(s) 12 may be regulated accordingly to control in real-time the occlusion of the vessel, and thereby ensure a desired amount of fluid flow is achieved based on the selected degree of inflation. The sensor(s) S1 or S2 may be of the piezo electrical or optical type, as examples, but other forms may be used. Instead of using automated control, the sensor data may also be used to manually adjust the balloon inflation, such as by displaying pressure or flow values to an operator in control or one or more indeflators. Any or all of the parts of the disclosed apparatus may be fully or partially provided with an anti-thrombotic coating to aid in reducing clotting.

Summarizing, this disclosure may be considered to relate to the following items:
1. An apparatus for selectively occluding a vessel, comprising:
   a catheter shaft including a first inflation lumen and a second inflation lumen; and
   a first group of inflatable balloons in communication with the first inflation lumen and a second group of inflatable balloons in communication with the second inflation lumen.
2. The apparatus of item 1, wherein the first and second groups of inflatable balloons comprise peripheral balloons surrounding a central balloon.
3. The apparatus of item 2, wherein the shaft includes a third inflation lumen in communication with the central balloon.
4. The apparatus of item 2, wherein the central balloon is in communication with the first inflation lumen.
5. The apparatus of any of items 1-4, wherein the central balloon is compliant and the peripheral balloons are non-compliant, the central balloon is non-compliant and the peripheral balloons are compliant, or one or more of the peripheral balloons are compliant and one or more of the peripheral balloons are non-complaint.
6. The apparatus of any of items 1-5, wherein one or more of the balloons is oblong, spherical or tapered.
7. The apparatus of any of items 1-6, wherein there are between 1 and 20 balloons in the first and second groups of balloons.
8. The apparatus of any of items 1-7, wherein the balloons of the first group alternate in position with the balloons of the second group in a single cross section.
9. The apparatus of any of items 1-8, further including an external shell around the first and second groups of balloons.
10. An apparatus for selectively occluding a vessel, comprising:
   a catheter shaft supporting a plurality of spherical balloons in a single cross-section of the catheter.
11. The apparatus of item 10, wherein the plurality of spherical balloons comprise compliant balloons.
12. The apparatus of item 10 or item 11, wherein the plurality of spherical balloons comprise a central balloon surrounded by plurality of peripheral balloons.
13. The apparatus of item 10 or item 11, further including a central balloon surrounded by the plurality of spherical balloons.
14. The apparatus of any of items 10-13, wherein the catheter shaft includes a first inflation lumen and a second inflation lumen, and wherein at least a first one of the spherical balloons is in communication with the first inflation lumen and at least a second one of the spherical balloons in communication with the second inflation lumen.
15. An apparatus for selectively occluding a vessel, comprising:
   a catheter shaft supporting a plurality of balloons in a single cross-section of the catheter, at least one of the balloons comprising a tapered balloon having an apical region adapted to create a minimal point of contact with an inner wall of the vessel.
16. The apparatus of item 15, wherein the at least one balloon tapers continuously from a proximal end portion forming the apex to a distal end portion.
17. The apparatus of item 15 or item 16, wherein the catheter shaft includes a first inflation lumen and a second inflation lumen, and wherein at least a first one of the plurality of balloons is in communication with the first inflation lumen and at least a second one of the plurality of balloons is in communication with the second inflation lumen.
18. An apparatus for selectively occluding a vessel, comprising:
   a catheter shaft supporting an inflatable perfusion balloon; and
   a flow restrictor external to the inflatable perfusion balloon for selectively moving in a longitudinal direction relative to the inflatable perfusion balloon for restricting flow therethrough when the inflatable perfusion balloon is fully inflated.
19. The apparatus of item 18, wherein the flow restrictor comprises a cone for covering a proximal end of the perfusion balloon.
20. The apparatus of item 18 or item 19, wherein the perfusion balloon comprises a plurality of balloons in a single cross-section of the catheter.
21. The apparatus of any of items 18-20, wherein the flow restrictor is supported by a sheath overlying the catheter shaft.
22. An apparatus for selectively occluding a vessel, comprising:
   a catheter shaft supporting a plurality of inflatable balloons in tandem, each of the plurality of balloons having a different nominal diameter when inflated.

23. The apparatus of item 22, wherein the plurality of balloons comprise a first proximal balloon having a first diameter and a second distal balloon having a second diameter smaller than the first diameter.

24. The apparatus of item 22 or item 23, wherein the catheter shaft includes a first inflation lumen and a second inflation lumen, and wherein at least a first one of the plurality of balloons is in communication with the first inflation lumen and at least a second one of the plurality of balloons is in communication with the second inflation lumen.

25. The apparatus of any of items 22-24, further including a third balloon intermediate of the first and second balloons, the third balloon having a third diameter intermediate of the first and second diameters.

26. The apparatus of item 25, wherein the catheter shaft includes a first inflation lumen, a second inflation lumen, and a third inflation lumen, wherein at least a first one of the plurality of balloons is in communication with the first inflation lumen, at least a second one of the plurality of balloons is in communication with the second inflation lumen, and at least a third one of the plurality of inflatable balloons is in communication with the third inflation lumen.

27. An apparatus for selectively occluding first and second branched vessels, comprising:
a main catheter shaft comprising first and second inflatable balloons for independently occluding the first and second branched vessels.
28. The apparatus of item 27, further including a first auxiliary shaft at least partially within the main catheter shaft supporting the first inflatable balloon and a second auxiliary shaft at least partially within the main catheter supporting the second inflatable balloon.
29. The apparatus of item 27 or item 28, wherein the first and second balloons are serially arranged.
30. The apparatus of item 27 or item 28, wherein the first and second balloons are arranged in parallel.
31. An apparatus for selectively occluding a vessel, comprising:
   a catheter shaft including an inflatable balloon having an annular shape; and
   a flexible cone interconnecting an end portion of the shaft and an end portion of the inflatable balloon.
32. The apparatus of item 31, wherein the flexible cone comprises a proximal cone connecting a proximal portion of the shaft with a proximal end portion of the inflatable balloon.
33. The apparatus of item 31 or item 32 further including a distal cone connected between a distal portion of the shaft and a distal end of the inflatable balloon.
34. The apparatus of any of items 31-33, wherein the catheter shaft extends through a central passage of the inflatable annular balloon.
35. The apparatus of any of items 1-34, further including a controller for controlling an inflation status of one or more of the balloons based on a sensor for sensing a parameter of a flow in the vessel.
36. The apparatus of item 35, wherein the sensor is located adjacent a distal end of the catheter shaft.
37. The apparatus of item 35, wherein the sensor is located adjacent a proximal end of the one or more balloons.
38. The apparatus of item 35, wherein the sensor comprises a first sensor located adjacent a proximal end of the one or more balloons and a second sensor located adjacent a distal end of the catheter shaft.
39. A method for selectively occluding a vessel, comprising:
   selectively inflating a first group inflatable balloons in the vessel independently of a second group of inflatable balloons.
40. The method of item 39, wherein the inflating step comprises inflating one or more peripheral balloons surrounding a central balloon.
41. The method of item 39 or item 40, further including the step of inflating the central balloon.
42. The method of any of items 39-41, further including the step of selectively moving a flow restrictor toward or away from a proximal end of the inflatable balloons.
43. A method for selectively occluding a vessel, comprising:
   moving a flow restrictor external to a perfusion balloon in a longitudinal direction relative to the inflatable perfusion balloon for restricting flow therethrough when the inflatable perfusion balloon is fully inflated.
44. A method for selectively occluding a vessel, comprising:
   selectively inflating a plurality of inflatable balloons connected to a catheter shaft in tandem, each of the plurality of balloons having a different nominal diameter when inflated.
45. A method for selectively occluding first and second branched vessels, comprising:
   positioning a main catheter shaft comprising first and second independently maneuverable inflatable balloons for occluding each of the first and second branched vessels.
46. The method of item 45, further comprising positioning a first auxiliary shaft within the main catheter shaft supporting the first inflatable balloon in the first branch vessel and positioning a second auxiliary within the main catheter supporting the second inflatable balloon in the second branch vessel.
47. A method for selectively occluding a vessel, comprising:
   expanding a flexible cone interconnecting an end portion of a catheter shaft and an end portion of an inflatable annular balloon.
48. The method of item 47, wherein the flexible cone is a proximal cone, and the method further including expanding a distal cone connected between a distal portion of the shaft and a distal end of the inflatable annular balloon.
49. The method of any of items 39-48, further including the step of controlling an inflation status of the one or more balloons based on a sensed parameter of a fluid flow within a vessel.

As used herein, the following terms have the following meanings:

“A”, “an”, and “the” as used herein refers to both singular and plural referents unless the context clearly dictates otherwise. By way of example, “a compartment” refers to one or more than one compartment.

“About,” “substantially,” or “approximately,” as used herein referring to a measurable value, such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows, e.g., component or the like does not exclude or preclude the presence of additional, non-recited components, features, element, members, steps, known in the art or disclosed therein.

Although the invention is described in conjunction with specific embodiments, many alternatives, modifications, and variations will be apparent to those skilled in the art. Accordingly, this disclosure embraces all such alternatives, modifications, and variations that fall within the spirit and scope of the appended claims. It is to be fully understood that certain aspects, characteristics, and features, of the invention, which are, for clarity, illustratively described and presented in the context or format of a plurality of separate embodiments, may also be illustratively described and presented in any suitable combination or sub-combination in the context or format of a single embodiment. Conversely, various aspects, characteristics, and features, of the invention which are illustratively described and presented in combination or sub-combination in the context or format of a single embodiment may also be illustratively described and presented in the context or format of a plurality of separate embodiments.

1. An apparatus for selectively occluding a vessel, comprising:
a catheter shaft including a first inflation lumen and a second inflation lumen; and
a first group of inflatable balloons in communication with the first inflation lumen and a second group of inflatable balloons in communication with the second inflation lumen.

2. The apparatus of claim 1, wherein the first and second groups of inflatable balloons comprise peripheral balloons surrounding a central balloon.

3. The apparatus of claim 2, wherein the catheter shaft includes a third inflation lumen in communication with the central balloon.

4. The apparatus of claim 2, wherein the central balloon is in communication with the first inflation lumen.

5. The apparatus of claim 2, wherein the central balloon is compliant and the peripheral balloons are non-compliant.

6. The apparatus of claim 1, wherein one or more of the balloons is oblong, spherical or tapered.

7. The apparatus of claim 1, wherein there are between 1 and 20 balloons in the first and second groups of balloons.

8. The apparatus of claim 1, wherein the balloons of the first group alternate in position with the balloons of the second group in a single cross section.

9. The apparatus of claim 1, further including an external shell around the first and second groups of balloons.

10. An apparatus for selectively occluding a vessel, comprising:
a catheter shaft supporting a plurality of balloons in a single cross-section, at least one of the balloons having a generally spherical shape.

11. The apparatus of claim 10, wherein the plurality of balloons comprise compliant balloons.

12. The apparatus of claim 10, wherein the plurality of balloons comprise a central balloon surrounded by a plurality of peripheral balloons.

13. The apparatus of claim 10, further including a central balloon surrounded by the plurality of balloons.

14. The apparatus of claim 10, wherein the catheter shaft includes a first inflation lumen and a second inflation lumen, and wherein at least a first one of the spherical balloons is in communication with the first inflation lumen and at least a second one of the spherical balloons in communication with the second inflation lumen.

15. An apparatus for selectively occluding a vessel, comprising:
a catheter shaft supporting a plurality of balloons in a single cross-section of the catheter, at least one of the balloons comprising a tapered balloon having an apical region adapted to create a minimal point of contact with an inner wall of the vessel. The apparatus of claim 15, wherein the at least one balloon tapers continuously from a proximal end portion forming the apex to a distal end portion.

17. The apparatus of claim 15, wherein the catheter shaft includes a first inflation lumen and a second inflation lumen, and wherein at least a first one of the plurality of balloons is in communication with the first inflation lumen and at least a second one of the plurality of balloons is in communication with the second inflation lumen.

18. An apparatus for selectively occluding a vessel, comprising:
a catheter shaft supporting an inflatable perfusion balloon; and
a flow restrictor external to the perfusion balloon for selectively moving in a longitudinal direction relative to the inflatable perfusion balloon for restricting flow therethrough when the inflatable perfusion balloon is fully inflated.

19. The apparatus of claim 18, wherein the flow restrictor comprises a cone for covering a proximal end of the perfusion balloon.

20. The apparatus of claim 18, wherein the perfusion balloon comprises a plurality of balloons in a single cross-section of the catheter.

21-49. (canceled)
